(19) United States
(12) Patent Application Publication
(10) Pub. No.: US 2018/0096191 A1
(43) Pub. Date: Apr. 5, 2018

(54) METHOD AND SYSTEM FOR AUTOMATED BRAIN TUMOR DIAGNOSIS USING IMAGE CLASSIFICATION

(71) Applicant: Siemens Aktiengesellschaft, Munich (DE)

(72) Inventors: Shaohua Wan, Beijing (CN); Shanhui Sun, Princeton, NJ (US); Subhabrata Bhattacharya, Seattle, WA (US); Terrence Chen, Princeton, NJ (US); Ali Kamen, Skillman, NJ (US)

(73) Assignee: Siemens Aktiengesellschaft, Munich (DE)

(21) Appl. No.: 15/559,264

(22) PCT Filed: Mar. 24, 2016
(86) PCT No.: PCT/US16/23929

§ 371 Date: Sep. 18, 2017

Related U.S. Application Data
(60) Provisional application No. 62/139,016, filed on Mar. 27, 2015.

(51) Int. Cl. G06K 9/00; G06K 9/62; G06T 7/00

(52) U.S. Cl. CPC G06K 9/00147; A61B 1/00009; G06T 7/0012; G06K 9/6255

(57) ABSTRACT

A method and system for classifying tissue endomicroscopy images are disclosed. Local feature descriptors are extracted from an endomicroscopy image. Each of the local feature descriptors is encoded using a learnt discriminative dictionary. The learnt discriminative dictionary includes class-specific sub-dictionaries and penalizes correlation between bases of sub-dictionaries associated with different classes. Tissue in the endomicroscopy image is classified using a trained machine learning based classifier based on the coded local feature descriptors encoded using a learnt discriminative dictionary.

This application claims the benefit of U.S. Provisional Application No. 62/139,016, filed Mar. 27, 2015, the disclosure of which is herein incorporated by reference.

### TECHNICAL FIELD

The present invention relates to classifying different types of tissue in medical image data using machine learning based image classification, and more particularly to automatic brain tumor diagnosis using machine learning based image classification.

### BACKGROUND OF THE INVENTION

Cancer is a major health problem throughout the world. Early diagnosis of cancer is crucial to the success of cancer treatments. Traditionally, pathologists acquire histopathological images of biopsies sampled from patients, examine the histopathological images under microscopy, and make judgments as to a diagnosis based on their knowledge and experience. Unfortunately, intraoperative fast histopathology is often not sufficiently informative for pathologists to make an accurate diagnosis. Biopsies are often non-diagnostic and yield inconclusive results for various reasons. Such reasons include sampling errors, in which the biopsy may not originate from the most aggressive part of a tumor. Furthermore, the tissue architecture of the tumor can be altered during the specimen preparation. Other disadvantages include the lack of interactivity and a waiting time of about 30-45 minutes for the diagnosis result.

Confocal laser endomicroscopy (CLE) is a medical imaging technique that provides microscopic information of tissue in real-time on cellular and subcellular levels. Thus, CLE can be used to perform an optical biopsy and pathologists are able to access image directly in the surgery room. However, manual judgement as to a diagnosis may be subjective and variable across different pathologists. In addition, due to large amounts of image data acquired, the diagnosis task based on the optical biopsy can be a significant burden for pathologists. A computer-aided method for automated tissue diagnosis is desirable to reduce the burden and to provide quantitative numbers to support a pathologist's final diagnosis.

### BRIEF SUMMARY OF THE INVENTION

The present invention provides a method and system for automated classification of different types of tissue in medical images using machine learning based image classification. Embodiments of the present invention reconstruct image features of input endomicroscopy images using a learnt discriminative dictionary and classify the tissue in the endomicroscopy images based on the reconstructed image features using a trained classifier. Embodiments of the present invention utilize a dictionary learning algorithm that explicitly learns class-specific sub-dictionaries that minimize the effect of commonality among the sub-dictionaries. Embodiments of the present invention can be used to distinguish between glioblastoma and meningioma and classify brain tumor tissue in confocal laser endomicroscopy (CLE) images as malignant or benign.

In one embodiment of the present invention, local feature descriptors are extracted from an endomicroscopy image. Each of the local feature descriptors is encoded using a learnt discriminative dictionary. The learnt discriminative dictionary includes class-specific sub-dictionaries and penalizes correlation between bases of sub-dictionaries associated with different classes. Tissue in the endomicroscopy image is classified using a trained machine learning based classifier based on coded local feature descriptors resulting from encoding each of the local feature descriptors using a learnt discriminative dictionary.

These and other advantages of the invention will be apparent to those of ordinary skill in the art by reference to the following detailed description and the accompanying drawings.

### BRIEF DESCRIPTION OF THE DRAWINGS

FIG. 1 illustrates an example of a system for acquiring and processing endomicroscopy images according to an embodiment of the present invention;

FIG. 2 illustrates exemplary CFE images of brain tumor tissue;

FIG. 3 illustrates an overview of a pipeline for the online image classification for classifying tissue in endomicroscopy images according to an embodiment of the present invention;

FIG. 4 illustrates a method of learning a discriminative dictionary and training a classifier for classifying tissue in endomicroscopy images according to an embodiment of the present invention;

FIG. 5 illustrates a method for classifying tissue in one or more endomicroscopy images according to an embodiment of the present invention; and

FIG. 6 is a high-level block diagram of a computer capable of implementing the present invention.

### DETAILED DESCRIPTION

The present invention relates to automated classification of different types of tissue in medical images using a machine learning based image classification. Embodiments of the present invention can be applied to endomicroscopy images of brain tumor tissue for automated brain tumor diagnosis. Embodiments of the present invention are described herein to give a visual understanding of the method for automated classification of tissue in medical images. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

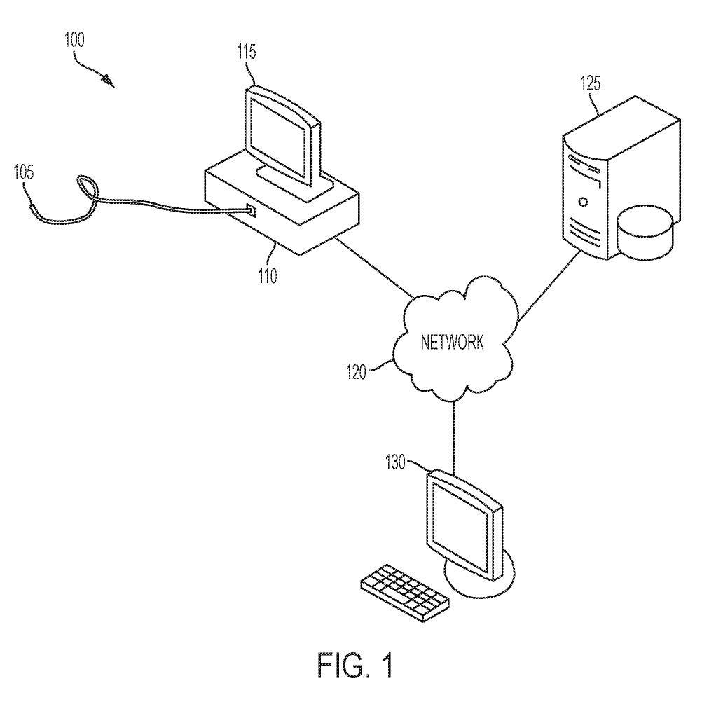

FIG. 1 illustrates an example of a system 100 for acquiring and processing endomicroscopy images according to an embodiment of the present invention. Briefly, endomicroscopy is a technique for obtaining histology-like images from inside the human body in real-time through a process known as “optical biopsy.” The term “endomicroscopy” generally refers to fluorescence confocal microscopy, although multi-photon microscopy and optical coherence tomography have also been adapted for endoscopic use and may be likewise used in various embodiments. Non-limiting examples of commercially available clinical endomicroscopes include the Pentax ISC-1000/EC3870CIK and Cellvizio (Mauna Kea Technologies, Paris, France). The main applications have traditionally been in imaging the gastro-intestinal tract, particularly for the diagnosis and characterization of Barrett's Esophagus, pancreatic cysts and colorectal lesions. The diagnostic spectrum of confocal endomicroscopy has recently expanded from screening and surveillance for colorectal cancer towards Barrett's esophagus, Helicobacter pylori associated gastritis and early gastric cancer. Endomicroscopy enables subsurface analysis of the gut mucosa and in-vivo histology during ongoing endoscopy in full resolution by point scanning laser fluorescence analysis. Cellular, vascular and connective structures can be seen in detail. Confocal laser endomicroscopy (CLE) provides detailed images of tissue on a cellular and sub-cellular level. In addition to being applied in the gastro-intestinal tract, endomicroscopy may also be applied to brain surgery where identification of malignant (Glioblastoma) and benign (Meningioma) tumors from normal tissues is clinically important.

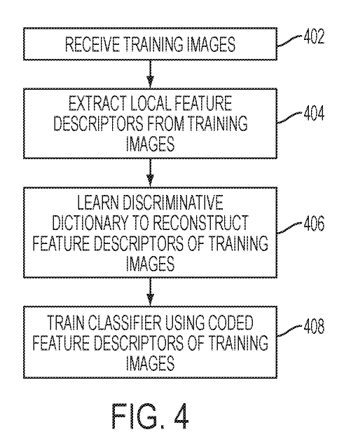

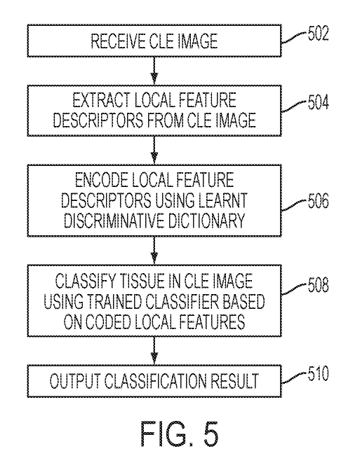

In the example of FIG. 1, a group of devices are configured to perform Confocal Laser Endomicroscopy (CLE). These devices include a Probe 105 operably coupled to an Imaging Computer 110 and an Imaging Display 115. In FIG. 1, Probe 105 is a confocal miniature probe. However, it should be noted that various types of miniature probes may be used, including probes designed for imaging various fields of view, imaging depths, distal tip diameters, and lateral and axial resolutions. The Imaging Computer 110 provides an excitation light or laser source used by the Probe 105 during imaging. Additionally, the Imaging Computer 110 may include imaging software to perform tasks such as recording, reconstructing, modifying, and/or export images gathered by the Probe 105. The Imaging Computer 110 may also be configured to perform a cell classification method, discussed in greater detail below with respect to FIG. 5, as well as training processes for learning a discriminative dictionary and training a machine learning based classifier, discussed in greater detail below with respect to FIG. 4.

A foot pedal (not shown in FIG. 1) may also be connected to the Imaging Computer 110 to allow the user to perform functions such as, for example, adjusting the depth of confocal imaging penetration, start and stop image acquisition, and/or saving image either to a local hard drive or to a remote database such as Database Server 125. Alternatively or additionally, other input devices (e.g., computer, mouse, etc.) may be connected to the Imaging Computer 110 to perform these functions. The Imaging Display 115 receives images captured by the Probe 105 via the Imaging Computer 110 and presents those images for view in the clinical setting.

Continuing with the example of FIG. 1, the Imaging Computer 110 is connected (either directly or indirectly) to a Network 120. The Network 120 may comprise any computer network known in the art including, without limitation, an intranet or internet. Through the Network 120, the Imaging Computer 110 can store images, videos, or other related data on a remote Database Server 125. Additionally a User Computer 130 can communicate with the Imaging Computer 110 or the Database Server 125 to retrieve data (e.g., images, videos, or other related data) which can then be processed locally at the User Computer 130. For example, the User Computer 130 may retrieve data from either Imaging Computer 110 or the Database Server 125 and use such to perform the cell classification method discussed below in FIG. 5 and/or the training processes for learning a discriminative dictionary and training a machine learning based classifier discussed below in FIG. 4.

Although FIG. 1 shows a CLE-based system, in other embodiments, the system may alternatively use a DHM imaging device. DHM, also known as interference phase microscopy, is an imaging technology that provides the ability to quantitatively track sub-nanometric optical thickness changes in transparent specimens. Unlike traditional digital microscopy, in which only intensity (amplitude) information about a specimen is captured, DHM captures both phase and intensity. The phase information, captured as a hologram, can be used to reconstruct extended morphological information (e.g., depth and surface characteristics) about the specimen using a computer algorithm. Modern DHM implementations offer several additional benefits, such as fast scanning/data acquisition speed, low noise, high resolution and the potential for label-free sample acquisition. While DHM was first described in the 1960s, instrument size, complexity of operation and cost has been major barriers to widespread adoption of this technology for clinical or point-of-care applications. Recent developments have attempted to address these barriers while enhancing key features, raising the possibility that DHM could be an attractive option as a core, multiple impact technology in healthcare and beyond.

An image based retrieval approach has been proposed to perform endomicroscopic image recognition tasks. In such an approach, classification is performed by querying an image database with Bag of feature Words (BoW)-based image representation and the most similar images from the database are retrieved. However, this approach requires large amounts of storage space which may be unfeasible for large database sizes. Embodiments of the present invention encode feature descriptors extracted from endomicroscopy images using learnt task-specific dictionaries.

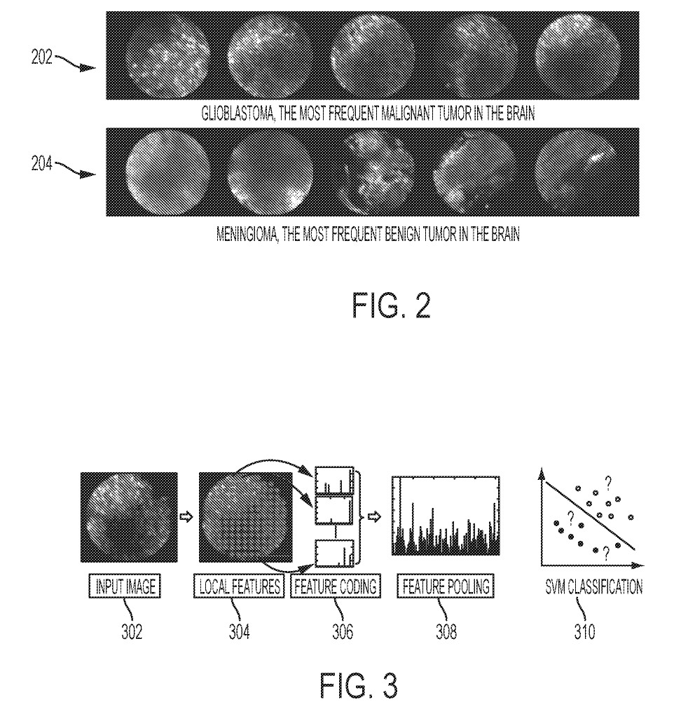

Embodiments of the present invention utilize an automated machine learning based framework to classify endomicroscopy images to different tissue types. This framework has three stages: (1) offline dictionary learning; (2) offline classifier training; and (3) online image classification. Embodiments of the present invention apply this image classification framework to automated brain tumor diagnosis to distinguish between two types of brain tumors: Glioblastoma and Meningioma. It is possible to learn an overcomplete dictionary to approximate feature descriptors of a given endomicroscopy image. However, the present inventors have observed that, despite the highly discriminative features contained by the images of different categories of tissue (e.g., glioblastoma and meningioma), these images may also share common patterns which do not contribute to the image recognition task. Another challenge in distinguishing glioblastoma and meningioma is the large intra-class variance and small inter-class commonality of the two types of brain tumors. FIG. 2 illustrates exemplary CFE images of brain tumor tissue. As shown in FIG. 1, row 202 shows CFE images of glioblastoma, the most frequent malignant type of brain tumor, and row 204 shows CFE images of meningioma, the most frequent benign type of brain tumor. As can be seen in FIG. 2, there is a great variability between images from the same class of brain tumor. In addition, the decision boundary between the two types of brain tumors is not clear, as granular and homogenous patterns are mixed in both classes.

To solve the above described challenges and improve the performance of the dictionary based classification pipeline, embodiments of the present invention learns a discriminative dictionary using a dictionary learning algorithm that explicitly learns class-specific sub-dictionaries that minimize the effect of commonality among the sub-dictionaries. The learnt discriminative dictionary can be used with any dictionary-code based coding methods, such as BoW, sparse coding, and locality-constraint coding. In addition, new coding methods fully utilizing the learnt discriminative dictionary are described herein.

In an advantageous embodiment, automated machine-learning based classification of tissue in endomicroscopy images is performed in three stages of off-line unsupervised codebook (dictionary) learning, off-line supervised classifier training, and on-line image or video classification. FIG. 3 illustrates an overview of a pipeline for the online image classification for classifying tissue in endomicroscopy images according to an embodiment of the present invention. As shown in FIG. 3, the pipeline for classifying tissue in an endomicroscopy image includes acquisition of an input image 302, local feature extraction 304, feature coding 306, feature pooling 308, and classification 210. Local feature points are detected on the input image, and feature descriptors, such as scale invariant feature transform (SIFT) or histograms of oriented gradients (HOG) feature descriptors, are extracted at each feature point. A learnt codebook or dictionary with K entries is applied to quantize each feature descriptor and generate a “code” layer. The terms “codebook” and “dictionary” are used interchangeably herein. It is possible to generate the dictionary using a K-means clustering method. However, in an advantageous embodiment of the present invention, a discriminative dictionary is generated using a dictionary learning method that explicitly learns class-specific sub-dictionaries that minimize the effect of commonality among the sub-dictionaries. For the supervised classification, each feature descriptor is then converted an [symbol] ^{K} code and the coded feature descriptors for the input image are pooled to yield and image representation. A classifier is trained to classify endomicroscopy images based on coded feature descriptors, and the trained classifier is applied to the pooled coded feature descriptors representing the input image to classify the tissue in the input image. In possible embodiments, a support vector machine (SVM) or random forest classifier is used, but the present invention is not limited to any specific classifier and any type of machine learning based classifier may be used.

FIG. 4 illustrates a method of learning a discriminative dictionary and training a classifier for classifying tissue in endomicroscopy images according to an embodiment of the present invention. The method of FIG. 4 can be performed offline to learn a discriminative dictionary and trained a machine learning classifier prior to online image classification using the learnt discriminative dictionary and trained classifier to classify tissue in an input endomicroscopy image. Referring to FIG. 4, at step 402, training images are received. The training images are endomicroscopy images of particular types of tissue and a class corresponding to the type of tissue is known for each training image. For example, the training can be divided into two classes corresponding to malignant and benign tissue. It is also possible that the training images be divided into three or more classes corresponding to different types of tissue. In an advantageous implementation, the training images are CLE images. In an exemplary embodiment, the training images can be CLE images of brain tumors, and each training image can be classified as glioblastoma or meningioma. The training images can be received by loading the training images from an image database.

At step 404, local feature descriptors are extracted from the training images. In a possible implementation, local feature points are detected on each training image, and local feature descriptors are extracted at each of the feature points on each training image. Various techniques may be applied for feature extraction. For example, feature descriptors such as, Scale Invariant Feature Transform (SIFT), Local Binary Pattern (LBP), Histogram of Oriented Gradient (HOG), and Gabor features, can be extracted at each of a plurality of points in each training image. Each technique may be configured based on the clinical application and other user-desired characteristics of the results. For example, the SIFT feature descriptor is a local feature descriptor that has been used for a large number of purposes in computer vision. It is invariant to translations, rotations and scaling transformations in the image domain and robust to moderate perspective transformations and illumination variations. The SIFT descriptor has been proven very useful in practice for image matching and object recognition under real-world conditions. In one exemplary implementation, dense SIFT descriptors of 20×20 pixel patches computed over a grid with spacing of 10 pixels are utilized. Such dense image descriptors may be used to capture uniform regions in cellular structures such as low-contrast regions in case of meningioma.

In another possible embodiment, rather than using human-designed feature descriptors, machine learning techniques may be used to learn filters that are discriminatively valuable from the training images. These machine-learning techniques may use various feature detection techniques including, without limitation, edge detection, corner detection, blob detection, ridge detection, edge direction, change in intensity, motion detection, and shape detection.

Returning to FIG. 4, at step 406, a discriminative dictionary is learned that can reconstruct the local feature descriptors of the training images as a sparse linear combination of bases in the discriminative dictionary. According to an advantageous embodiment, the discriminative dictionary includes class-specific sub-dictionaries that minimize the effect of commonality among the sub-dictionaries. For example, in the case in which the training images are CLE images of glioblastoma and meningioma brain tumors, sub-dictionaries corresponding to each class (i.e., glioblastoma and meningioma) are learned. The learning method minimizes an error between the feature descriptors of the training images and the reconstructed feature descriptors using the discriminative dictionary while considering both the global dictionary and the individual class representations (sub-dictionaries) within the dictionary.

Given a training set Y=[y₁, . . . , y_{N}]∈ [symbol] ^{M×N}, traditional dictionary learning aims to learn a dictionary of bases that best reconstruct the training examples:

$$\begin{matrix}min \\ D , X\end{matrix} {\sum }_{i = 1}^{N} {‖ {y}_{i} - {Dx}_{i} ‖}_{2}^{2} + λ {‖ {x}_{i} ‖}_{1} \qquad ( 1 )$$

where D=[d₁, . . . , d_{K}]∈ [symbol] ^{M×K} is the dictionary with K bases, xᵢ∈ [symbol] ^{K} are the reconstruction coefficients for yᵢ, ∥•∥₁ denotes the l₁-norm that promotes the sparsity of the reconstruction coefficients, and λ is a tuning parameter. Different from K-means clustering that assigns each training example to the nearest cluster center, Equation (1) learns an overcomplete dictionary D and represents each training example as a sparse linear combination of the bases in the dictionary.

To learn a dictionary that is well-suited for supervised classification tasks, class-specific dictionary learning methods have been proposed that learn a sub-dictionary for each class. For example, such a dictionary learning method can be formulated as:

$$\begin{matrix}min \\ D , X\end{matrix} {\sum }_{c = 1}^{C} {‖ {Y}_{c} - {DX}_{c} ‖}_{2}^{2} + λ {‖ {X}_{c} ‖}_{1} \qquad ( 2 )$$

where C is the number of classes, Y_{c}=[y₁^{c}, . . . , y_{Nc}^{c}], X_{c}=[x₁^{c}, . . . , x_{Nc}^{c}], and D_{c}=[d₁^{c}, . . . , d_{Kc}^{c}] are the training set, reconstruction coefficients, and sub-dictionary for the class c, respectively. However, the sub-dictionaries learned using Equation (2) typically share common (correlated) bases. Thus, the dictionary D may not be sufficiently discriminative for classification tasks, and the sparse representation will be sensitive to variations in features.

According to an advantageous embodiment of the present invention, a discriminative dictionary is learned by learning high-order couplings between the feature representations of images in the form of a set of class-specific sub-dictionaries under elastic net regularization, which is formulated as:

$$\begin{matrix}min \\ D , X\end{matrix} {\sum }_{c = 1}^{C} [ {‖ {Y}_{c} - {DX}_{c} ‖}_{2}^{2} + {‖ {Y}_{c} - {D}_{∈ c} {X}_{c} ‖}_{2}^{2} + {‖ {D}_{∉ c} {X}_{c} ‖}_{2}^{2} + {λ}_{1} {‖ {X}_{c} ‖}_{1} + {λ}_{2} {‖ {X}_{c} ‖}_{2}^{2} ] \\ s . t . {‖ {d}_{k} ‖}_{2}^{2} = 1 , ∀ k = 1 , … , K \qquad ( 3 )$$

where D_{∈c}=[0, . . . , D_{∈C}, . . . 0] and D_{∉}=D−D_{∈c}. The term ∥Y_{c}−DX_{c}∥₂² minimizes the global reconstruction residual of the training examples using the whole dictionary. The term ∥Y_{c}—D_{∈c}X_{c}∥₂² minimizes the reconstruction residual of the training examples of class c using the c^{th} sub-dictionary. Accordingly, the minimization problem of Equation (3) learns dictionary bases D and reconstruction coefficients X to minimize the global residual for reconstruction of the training examples of a specific class from the all of dictionary bases as well as the residual for reconstruction of the training examples of the specific class from only bases of the sub-dictionary associated with that class, while penalizing the use of bases of sub-dictionaries not associated with the particular class in reconstruction training examples of that class. The term ∥D_{∉c}X_{c}∥₂² penalizes the reconstruction of training examples using sub-dictionaries from different classes. ë₁∥X_{c}∥₁+ë₂∥X_{c}∥₂² is the elastic net regularizer, where ë₁ and ë₂ are tuning parameters.

The elastic net regularizer is a weighted sum of the of the l₁-norm and the l₂-norm of the reconstruction coefficients. Compared to a pure l₁-norm regularizer, the elastic net regularizer allows the selection of groups of correlated features even if the group is not known in advance. In addition to enforcing the grouped selection, the elastic net regularizer is also crucial to the stability of the spare reconstruction coefficients with respect to the input training examples. The incorporation of the elastic net regularizer to enforce a group sparsity constraint provides the following benefits class-specific dictionary learning. First, the intra-class variations among features can be compressed since features from the same class tend to be reconstructed by bases within the same group (sub-dictionary). Second, the influence of correlated atoms (bases) from different sub-dictionaries can be minimized since their coefficients tend to be zero or non-zero simultaneously. Third, possible randomness in coefficients distribution can be removed since coefficients have group clustered sparse characteristics.

The discriminative dictionary D is learned by optimizing Equation (3). The optimization of Equation (3) can be iteratively solved by optimizing over D and X while fixing the other. D and X can be initialized using preset values. After fixing the dictionary D, the coefficients vector xⱼ^{c} (i.e., the coefficient vector of the j-th example in the c-th class) can be calculated by solving the following convex problem:

$$\begin{matrix}min \\ {x}_{j}^{c}\end{matrix} {‖ {s}_{j}^{c} - {\hat{D}}_{c} {x}_{j}^{c} ‖}_{2}^{2} + \ddot{e} \vert {x}_{j}^{c} \vert \qquad ( 4 )$$

where

sⱼ^{C}=[yⱼ^{c};yⱼ^{c};0; . . . ;0] (5a)

\hat{D}_{c}=[D;D_{∈c};D_{∉c};\sqrt{ë₂}I] (5b)

where I∈ [symbol] ^{K×K} is an identity matrix. In an advantageous implementation, the Alternating Direction Method of Multipliers (ADMM) procedure can be used to solve Equation (4). While the dictionary is fixed D, equation (4) is solved to optimize the coefficient vectors for all training examples in all classes.

The reconstruction coefficients are the fixed and with the reconstruction coefficients fixed, bases (atoms) in the dictionary are updated. In an advantageous embodiment, the sub-dictionaries are updated class by class. In other words, while updating the sub-dictionary D_{c}, all other sub-dictionaries will be fixed. Terms that are independent of the current sub-dictionary can then be omitted from the optimization. Thus the objective function for updating the sub-dictionary D_{c} can be expressed as:

$$\begin{matrix}min \\ {D}_{c}\end{matrix} {‖ {Y}_{c} - {DX}_{c} ‖}_{2}^{2} + {‖ {Y}_{c} - {D}_{∈ c} {X}_{c} ‖}_{2}^{2} \qquad ( 6 )$$

Analytical solutions exist for Equation (6). In particular, Equation (6) can be solved using the following analytical solution:

$${D}_{∈ c} = \frac{1}{2} {( {X}_{c} {X}_{c}^{T} )}^{- 1} {X}_{c} ( 2 {Y}_{c}^{T} - {X}_{c}^{T} {D}_{∉ c}^{T} ) . \qquad ( 7 )$$

Equation (6) can be solved for each sub-dictionary using the analytical solution in Equation (7) in order to update the dictionary bases for each sub-dictionary. The updating of the coefficients and dictionary bases can be iterated until the dictionary bases and/or reconstruction coefficients converge or until a preset number of iterations are performed. In an exemplary embodiment, a discriminative dictionary having two sub-dictionaries, one associated with a glioblastoma (malignant) class and one associated with a meningioma (benign) class, is learned for reconstructing local feature descriptors extracted from training images in the glioblastoma and meningioma classes.

Returning to FIG. 4, at step 408 a classifier is trained using the coded feature descriptors of the training images. The classifier is machine learning based classifier that is trained to classify an image into one of a plurality of classes corresponding to a type of tissue in the image based on coded feature descriptors extracted from the image and encoded using the learnt discriminative dictionary learned in step 406. Various methods can be used to encode each feature descriptor using the learnt dictionary. Such methods are described in greater detail below in connection with step 506 of FIG. 5. The coded feature descriptors for a particular training image can be pooled in order to generate an image representation of that training image. A machine learning based classifier is then trained based on the pooled coded feature descriptors for each of the training images and the known classes of the training images in order to classify images into the classes based on the pooled coded feature descriptors. For example, the machine learning based classifier may be implemented using a support vector machine (SVM), random forest classifier, or, k-nearest neighbors (k-NN) classifier, but the present invention is not limited thereto and other machine learning based classifiers may be used as well. In an exemplary embodiment, the classifier is trained to classify tissue in an endomicroscopy image as glioblastoma (malignant) or meningioma (benign) based on coded local feature descriptors extracted from the image.

FIG. 5 illustrates a method for classifying tissue in one or more endomicroscopy images according to an embodiment of the present invention. The method of FIG. 5 can be performed in real-time or near real-time during a surgical procedure to classify endomicroscopy images acquired during the surgical procedure. The method of FIG. 5 uses a learnt discriminative dictionary and a trained classifier that were learned/trained prior to the surgical procedure, for example using the method of FIG. 4. The method of FIG. 5 may be used to classify the tissue in individual endomicroscopy images or to classify the tissue in a sequence of endomicroscopy images (i.e., an endomicroscopy video stream).

Referring to FIG. 5, at step 502, an endomicroscopy image is received. For example, the endomicroscopy image may be a CLE image acquired using a CLE probe, such as probe 105 in FIG. 1. The endomicroscopy can an image frame received as part of an endomicroscopy video stream. In an advantageous embodiment, the endomicroscopy image can be received directly from a probe used to acquire the endomicroscopy image. In this case, the method of FIG. 5 can be performed in real-time or near real-time during a surgical procedure in which the endomicroscopy image is acquired. It is also possible that the endomicroscopy image is received by loading a previously acquired endomicroscopy from a storage or memory of a computer system performing the method of FIG. 5 or from a remote database. In an exemplary embodiment, the endomicroscopy image may be an endomicroscopy image of brain tumor tissue.

In a possible embodiment in which an endomicroscopy video stream is received, entropy-based pruning may be used to automatically remove image frames with low image texture information (e.g., low-contrast and contain little categorical information) that may not be clinically interesting or not suitable for image classification. This removal may be used, for example, to address the limited imaging capability of some CLE devices. Image entropy is a quantity which is used to describe the “informativeness” of an image, i.e., the amount of information contained in an image. Low-entropy images have very little contrast and large runs of pixels with the same or similar gray values. On the other hand, high entropy images have a great deal of contrast from one pixel to the next. For CLE images of glioblastoma and meningioma, low-entropy images contain a lot of homogeneous image regions, while high-entropy images are characterized by rich image structures. The pruning can be performed using an entropy threshold. This threshold may be set based on the distribution of the image entropy throughout the dataset of training images used for learning the discriminative dictionary and training the machine learning based classifier.

At step 504, local feature descriptors are extracted from the received endomicroscopy image. In an advantageous embodiment, a respective feature descriptor is extracted at each of a plurality of points on the endomicroscopy image, resulting in a plurality of local feature descriptors extracted from the endomicroscopy image. For example, a feature descriptor such as, Scale Invariant Feature Transform (SIFT), Local Binary Pattern (LBP), Histogram of Oriented Gradient (HOG), or Gabor features, can be extracted at each of a plurality of points in the endomicroscopy image. It is also possible that multiple of the above feature descriptors can be extracted at each of the plurality of points of the endomicroscopy image. In an exemplary implementation, the SIFT feature descriptor is extracted at each of a plurality of points of the endomicroscopy image. The SIFT feature descriptor is invariant to translations, rotations and scaling transformations in the image domain and robust to moderate perspective transformations and illumination variations. In one exemplary implementation, dense SIFT feature descriptors of 20×20 pixel patches computed over a grid with spacing of 10 pixels are extracted from the endomicroscopy image.

In another possible embodiment, rather than using human-designed feature descriptors, local features can be automatically extracted using filters that are learned from training images using machine learning techniques. These machine-learning techniques may use various feature detection techniques including, without limitation, edge detection, corner detection, blob detection, ridge detection, edge direction, change in intensity, motion detection, and shape detection.

At step 506, the local feature descriptors extracted from the endomicroscopy image are encoded using a learnt discriminative dictionary. In an advantageous embodiment, the learnt discriminative dictionary trained using the method of FIG. 4 is used to encode the local feature descriptors. A coding process is applied to each local feature descriptor extracted from the endomicroscopy image to convert that local feature descriptor into K-dimensional code xᵢ=[xᵢ₁, . . . , x_{iK}]∈ [symbol] ^{K} using the learnt discriminative dictionary of K bases, D=[d₁, . . . , d_{K}]∈ [symbol] ^{M×K}. The “code” for a particular local feature descriptor a vector of reconstruction coefficients for reconstructing that local feature descriptor as a linear combination of the bases of the learnt discriminative dictionary.

Various encoding schemes can be used to calculate the reconstruction coefficients x for an input local feature descriptor y using the learnt discriminative dictionary D. For example, the learnt discriminative dictionary can be used in place of a conventional dictionary in existing encoding schemes, such as BoW, sparse coding, or locality-constraint linear coding. Other encoding schemes to calculate the reconstruction coefficients x for an input local feature descriptor y using the learnt discriminative dictionary D are described herein, as well. Such feature encoding schemes are applied to each local descriptor extracted from the endomicroscopy image in order to determine the reconstruction coefficients for each local descriptor.

In an exemplary embodiment, reconstruction coefficients x for each local feature descriptor y can be calculated using feature encoding under the elastic-net regularizer. Encoding the local feature descriptor y under the elastic-net regularizer can be formulated as:

$$\begin{matrix}min \\ x\end{matrix} {‖ y - Dx ‖}_{2}^{2} + {\ddot{e}}_{1} {‖ x ‖}_{1} + {\ddot{e}}_{2} {‖ x ‖}_{2}^{2} . \qquad ( 8 )$$

Equation (8) can be re-written as:

$$\begin{matrix}min \\ x\end{matrix} {‖ \hat{y} - \hat{D} x ‖}_{2}^{2} + {\ddot{e}}_{1} {‖ x ‖}_{1} \qquad ( 9 )$$

where ŷ=[y;0], \hat{D}=[D;\sqrt{ë₂}I], and I∈ [symbol] ^{K×K} is an identity matrix. The ADMM optimization procedure can then be applied to optimize Equation (9) in order to calculate the reconstruction coefficients x.

In another exemplary embodiment, reconstruction coefficients x for each local feature descriptor y can be calculated using feature encoding by nearest centroid. In this embodiment, the local feature descriptor y can be encoded by the nearest dictionary basis, as follows:

$$\begin{matrix}\begin{matrix}min \\ x\end{matrix} {‖ y - Dx ‖}_{2}^{2} & ( 10 a ) \\ s . t . {1}^{T} x = 1 , {x}_{i} ∈ 0 , 1. & ( 10 b )\end{matrix}$$

In another exemplary embodiment, reconstruction coefficients x for each local feature descriptor y can be calculated using feature encoding under a locality-constrained linear regularizer. Encoding the local feature descriptor y under the locality-constrained linear regularizer can be formulated as:

$$\begin{matrix}\begin{matrix}min \\ x\end{matrix} {‖ y - Dx ‖}_{2}^{2} + \ddot{e} {‖ {b}^{T} x ‖}_{2}^{2} & ( 11 a ) \\ s . t . {1}^{T} x = 1 & ( 11 b )\end{matrix}$$

where b is a locality adaptor that gives a different weight for each dictionary basis proportional to its similarity to the input local feature descriptor y, i.e.,

$$b = exp ( \frac{dist ( y , D )}{ó} ) ,$$

where dist(y,D)=[dist(y,d₁), . . . , dist(y,d_{K})]^{T}, and ó is a tuning parameter used for adjusting the decay speed for the locality adaptor.

In another exemplary embodiment, reconstruction coefficients x for each local feature descriptor y can be calculated using feature encoding under locality-constrained sparse regularizer. Encoding the local feature descriptor y under the locality-constrained sparse regularizer can be formulated as:

$$\begin{matrix}\begin{matrix}min \\ x\end{matrix} {‖ y - Dx ‖}_{2}^{2} + \ddot{e} {‖ {b}^{T} x ‖}_{1} & ( 11 a ) \\ s . t . {1}^{T} x = 1 & ( 11 b )\end{matrix}$$

where b is a locality adaptor that gives a different weight for each dictionary basis proportional to its similarity to the input local feature descriptor y, i.e.,

$$b = exp ( \frac{dist ( y , D )}{ó} ) ,$$

where dist(y,D)=[dist(y,d₁), . . . , dist(y,d_{K})]^{T}, and ó is a tuning parameter used for adjusting the decay speed for the locality adaptor.

In another exemplary embodiment, reconstruction coefficients x for each local feature descriptor y can be calculated using feature encoding under a locality-constrained elastic-net regularizer. Encoding the local feature descriptor y under the locality-constrained elastic-net regularizer can be formulated as:

$$\begin{matrix}\begin{matrix}min \\ x\end{matrix} {‖ y - Dx ‖}_{2}^{2} + {\ddot{e}}_{1} {‖ {b}^{T} x ‖}_{1} + \ddot{{e}_{2}} {‖ {b}^{T} x ‖}_{2}^{2} & ( 11 a ) \\ s . t . {1}^{T} x = 1 & ( 11 b )\end{matrix}$$

where b is a locality adaptor that gives a different weight for each dictionary basis proportional to its similarity to the input local feature descriptor y, i.e.,

$$b = exp ( \frac{dist ( y , D )}{ó} ) ,$$

where dist(y,D)=[dist(y,d₁), . . . , dist(y,d_{K})]^{T}, and ó is a tuning parameter used for adjusting the decay speed for the locality adaptor.

Returning to FIG. 5, at step 508, the tissue in the endomicroscopy image is classified based on the coded local feature descriptors using a trained classifier. In an advantageous embodiment, the trained classifier is a machine learning based classifier trained using the method of FIG. 4. The trained classifier can be implemented using a linear support vector machine (SVM), random forest classifier, or k-nearest neighbors (k-NN) classifier, but the present invention is not limited thereto and other machine learning based classifiers may be used as well. The coded local feature descriptors, i.e., the reconstruction coefficients determined for each of the local feature descriptors, are input to the trained classifier and the trained classifier classifies the tissue in the endomicroscopy image based on these coded features. According to an advantageous embodiment of the present invention, since the dictionary learning method penalizes reconstruction of feature descriptors of training images in one class from dictionary bases in the sub-dictionaries other than the sub-dictionary associated with that class, local feature descriptors for an endomicroscopy image of a particular class will be reconstructed mostly using bases within the sub-dictionary associated with that class. Accordingly, the reconstruction parameters, which identify which bases in the discriminative dictionary are used to reconstruct each local feature descriptor, will have significant discriminative value in distinguishing between classes.

In an advantageous embodiment, the coded feature local descriptors (i.e., the reconstruction coefficients for each of the extracted local feature descriptors) for the endomicroscopy image can be pooled in order to generate an image representation of the endomicroscopy image prior to being input to the trained classifier. One or more feature pooling operations can be applied to summarize the coded local feature descriptors to generate a final image representation of the endomicroscopy image. For example, pooling techniques such as max-pooling, average-pooling, or a combination thereof, may be applied to the coded local feature descriptors. In a possible implementation, a combination of max-pooling and average-pooling operations can be used. For example, each feature map may be partitioned into regularly spaced square patches and a max-polling operation may be applied (i.e., the maximum response for the feature over each square patch may be determined). The max-pooling operation allows local invariance to translation. Then, the average of the maximum response may be calculated from the square patches, i.e. average pooling is applied after max-pooling. Finally, the image representation may be formed by aggregating feature responses from the average-pooling operation. Once the pooling is performed, the image representation generated by pooling the coded local feature descriptors for the endomicroscopy image is input to trained classifier and the trained classifier classifies the tissue in the endomicroscopy image based on the input image representation.

In an advantageous embodiment, the trained classifier classifies the tissue in the endomicroscopy image of a brain tumor as glioblastoma (malignant) or meningioma (benign). Further, in addition to classifying the tissue into one of a plurality of tissue classifications (e.g., glioblastoma or meningioma), the trained classifier may also calculate a classification score, which is a probability or confidence score regarding the classification result.

Returning to FIG. 5, at step 510, the classification result for the tissue in the endomicroscopy image is output. For example, class labeled identified for the tissue in the endomicroscopy image may be displayed on a display device of a computer system. For example, the class label may provide an indication of a specific type of tissue, such as glioblastoma or meningioma, or may provide an indication of whether the tissue in the endomicroscopy image is malignant or benign.

Although the method of FIG. 5 is described as classifying tissue in a single endomicroscopy image, the method of FIG. 5 can also be applied to an endomicroscopy video stream. As an endomicroscopy video stream is a sequence of endomicroscopy image frames, steps 502-508 can be repeated for multiple endomicroscopy image frames of an endomicroscopy video stream and a majority voting based classification scheme can be used to determine an overall classification of the tissue for the video stream based on the individual classification results of the tissue in each of the endomicroscopy image frames in the video sequence. Steps 502-508 can be repeated for a plurality of endomicroscopy image frames of a video stream acquired over a fixed length of time. The majority voting based classification then assigns an overall class label to the video stream using the majority voting result of the images within the video stream acquired over the fixed length time. The length of the window for a particular video stream may be configured based on user input. For example, the user may provide a specific length value or clinical setting which may be used to derive such a value. Alternatively, the length may be dynamically adjusted over time based on an analysis of past results. For example, if the user indicates that the majority voting classification is providing inadequate or sub-optimal results, the window maybe adjusted by modifying the window size by a small value. Over time, an optimal window length can be learned for the particular type of data being processed. Once the majority voting determines an overall classification for the tissue in the endomicroscopy video stream, step 510 is performed and the classification result for the video stream is output.

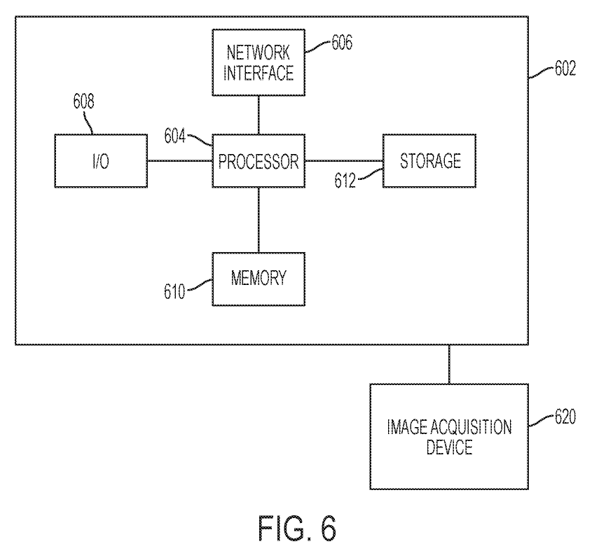

The above-described methods for learning a discriminative dictionary and training a machine learning based classifier, and automated classification of tissue in endomicroscopy images may be implemented on a computer using well-known computer processors, memory units, storage devices, computer software, and other components. A high-level block diagram of such a computer is illustrated in FIG. 6. Computer 602 contains a processor 604, which controls the overall operation of the computer 602 by executing computer program instructions which define such operation. The computer program instructions may be stored in a storage device 612 (e.g., magnetic disk) and loaded into memory 610 when execution of the computer program instructions is desired. Thus, the steps of the methods of FIGS. 3-5 may be defined by the computer program instructions stored in the memory 610 and/or storage 612 and controlled by the processor 604 executing the computer program instructions. An image acquisition device 620, such as a CLE probe, can be operably connected to the computer 602 to input image data to the computer 602. It is possible that the image acquisition device 620 and the computer 602 be directly connected or implemented as one device. It is also possible that the image acquisition device 620 and the computer 602 communicate wirelessly through a network. In a possible embodiment, the computer 602 can be located remotely with respect to the image acquisition device 620 and the some or all of the method steps described herein can be performed as part of a server or cloud based service. In this case, the method steps may be performed on a single computer or distributed between multiple networked and/or local computers. The computer 602 also includes one or more network interfaces 606 for communicating with other devices via a network. The computer 602 also includes other input/output devices 608 that enable user interaction with the computer 602 (e.g., display, keyboard, mouse, speakers, buttons, etc.). One skilled in the art will recognize that an implementation of an actual computer could contain other components as well, and that FIG. 6 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A method for classifying tissue in one or more endomicroscopy images, comprising:
extracting local feature descriptors from an endomicroscopy image;
encoding each of the local feature descriptors into a coded local feature descriptor using a learnt discriminative dictionary, wherein (a) each coded local feature descriptor comprises a vector of reconstruction coefficients for reconstructing the local feature descriptor as a linear combination of bases of the learnt discriminative dictionary (b) the learnt discriminative dictionary includes class-specific sub-dictionaries and penalizes correlation between bases of sub-dictionaries associated with different classes; and
applying a machine learning-based classifier to classify tissue in the endomicroscopy image into one of a plurality of classes based on the coded local feature descriptors.

2. The method of claim 1, wherein the endomicroscopy image is a confocal laser endomicroscopy (CLE) image acquired using a CLE probe.

3. The method of claim 1, further comprising:
learning the learnt discriminative dictionary based on local feature descriptors extracted from training images.

4. The method of claim 3, wherein learning the learnt discriminative dictionary based on local feature descriptors extracted from training images comprises:
learning the class-specific sub-dictionaries and reconstruction coefficients that, for each of a plurality of class, minimizes a total reconstruction residual of local feature descriptors extracted from training images of that class using all bases and a reconstruction residual of the local feature descriptors extracted from training images of that class using bases of the sub-dictionary associated with that class, and penalizes reconstruction of local feature descriptors extracted from training images of that class using bases of sub-dictionaries not associated with that class.

5. The method of claim 4, wherein learning the class-specific sub-dictionaries and reconstruction coefficients comprises:
learning the class-specific sub-dictionaries and reconstruction coefficients under elastic-net regularization.

6. The method of claim 5, wherein learning the class-specific sub-dictionaries and reconstruction coefficients comprises:
iteratively optimizing an objective function by:
updating reconstruction coefficients for the local feature descriptors extracted each training image of each class with a fixed discriminative dictionary, and
updating bases in each class-specific sub-dictionary with fixed reconstruction coefficients.

7. (canceled)

8. (canceled)

9. The method of claim 1, wherein encoding each of the local feature descriptors into the coded local feature descriptor using the learnt discriminative dictionary comprises:
determining the reconstruction coefficients to encode each of the local feature descriptors under an elastic-net regularizer using the learnt discriminative dictionary.

10. The method of claim 1, encoding each of the local feature descriptors into the coded local feature descriptor using the learnt discriminative dictionary comprises:
determining the reconstruction coefficients to encode each of the local feature descriptors by a nearest dictionary basis in the learnt discriminative dictionary.

11. The method of claim 1, wherein encoding each of the local feature descriptors into the coded local feature descriptor using the learnt discriminative dictionary comprises:
determining the reconstruction coefficients to encode each of the local feature descriptors under a locality-constrained linear regularizer using the learnt discriminative dictionary.

12. The method of claim 1, wherein encoding each of the local feature descriptors into the coded local feature descriptor using the learnt discriminative dictionary comprises:
determining the reconstruction coefficients to encode each of the local feature descriptors under a locality-constrained sparse regularizer using the learnt discriminative dictionary.

13. The method of claim 1, wherein encoding each of the local feature descriptors into the coded local feature descriptor using the learnt discriminative dictionary comprises:
determining the reconstruction coefficients to encode each of the local feature descriptors under a locality-constrained elastic-net regularizer using the learnt discriminative dictionary.

14. The method of claim 1, wherein the machine learning-based classifier is a support vector machine (SVM).

15. The method of claim 1, further comprising:
repeating the steps of extracting local feature descriptors, encoding each of the local feature descriptors using the learnt discriminative dictionary, and applying the machine learning-based classifier to classify the tissue in the endomicroscopy image for each of a plurality of endomicroscopy images in an endomicroscopy video stream; and
classifying the tissue in the endomicroscopy video stream based on the classification of the tissue in each of the plurality of endomicroscopy images in an endomicroscopy video stream.

16. The method of claim 1, wherein the endomicroscopy image is an endomicroscopy image of a brain tumor, and applying the machine learning-based classifier to classify the tissue in the endomicroscopy image comprises:
classifying the tissue in the endomicroscopy image as glioblastoma or meningioma using the machine learning-based classifier based on the coded local feature descriptors.

17. An apparatus for classifying tissue in one or more endomicroscopy images, comprising:
means for extracting local feature descriptors from an endomicroscopy image;
means for encoding each of the local feature descriptors into a coded local feature descriptor using a learnt discriminative dictionary, wherein (a) each coded local feature descriptor comprises a vector of reconstruction coefficients for reconstructing the local feature descriptor as a linear combination of bases of the learnt discriminative dictionary (b) the learnt discriminative dictionary includes class-specific sub-dictionaries and penalizes correlation between bases of sub-dictionaries associated with different classes; and
means for applying a machine learning-based classifier to classify tissue in the endomicroscopy image into one of a plurality of classes based on the coded local feature descriptors.

18. The apparatus of claim 17, wherein the endomicroscopy image is a confocal laser endomicroscopy (CLE) image acquired using a CLE probe.

19. The apparatus of claim 17, further comprising:
means for learning the learnt discriminative dictionary based on local feature descriptors extracted from training images.

20. The apparatus of claim 19, wherein the means for learning the learnt discriminative dictionary based on local feature descriptors extracted from training images comprises:
means for learning the class-specific sub-dictionaries and reconstruction coefficients that, for each of a plurality of class, minimizes a total reconstruction residual of local feature descriptors extracted from training images of that class using all bases and a reconstruction residual of the local feature descriptors extracted from training images of that class using bases of the sub-dictionary associated with that class, and penalizes reconstruction of local feature descriptors extracted from training images of that class using bases of sub-dictionaries not associated with that class.

21. (canceled)

22. (canceled)

23. The apparatus of claim 17, further comprising:
means for classifying tissue in an endomicroscopy video stream comprising a plurality of endomicroscopy images based on a classification of the tissue in individual ones of the plurality of endomicroscopy images in the endomicroscopy video stream.

24. The apparatus of claim 17, wherein the endomicroscopy image is an endomicroscopy image of a brain tumor, and the means for means for applying a machine learning-based classifier to classify tissue in the endomicroscopy image into one of a plurality of classes comprises:
means for classifying the tissue in the endomicroscopy image as glioblastoma or meningioma using the machine learning-based classifier based on the coded local feature descriptors.

25. A non-transitory computer readable medium storing computer program instructions for classifying tissue in one or more endomicroscopy images, the computer program instructions when executed by a processor cause the processor to perform operations comprising:
extracting local feature descriptors from an endomicroscopy image;
encoding each of the local feature descriptors into a coded local feature descriptor using a learnt discriminative dictionary, wherein (a) each coded local feature descriptor comprises a vector of reconstruction coefficients for reconstructing the local feature descriptor as a linear combination of bases of the learnt discriminative dictionary (b) the learnt discriminative dictionary includes class-specific sub-dictionaries and penalizes correlation between bases of sub-dictionaries associated with different classes; and
applying a machine learning-based classifier to classify tissue in the endomicroscopy image into one of a plurality of classes based on the coded local feature descriptors.

26. The non-transitory computer readable medium of claim 25, wherein the endomicroscopy image is a confocal laser endomicroscopy (CLE) image acquired using a CLE probe.

27. The non-transitory computer readable medium of claim 25, wherein the operations further comprise:
learning the learnt discriminative dictionary based on local feature descriptors extracted from training images.

28. The non-transitory computer readable medium of claim 27, wherein learning the learnt discriminative dictionary based on local feature descriptors extracted from training images comprises:
learning the class-specific sub-dictionaries and reconstruction coefficients that, for each of a plurality of class, minimizes a total reconstruction residual of local feature descriptors extracted from training images of that class using all bases and a reconstruction residual of the local feature descriptors extracted from training images of that class using bases of the sub-dictionary associated with that class, and penalizes reconstruction of local feature descriptors extracted from training images of that class using bases of sub-dictionaries not associated with that class.

29. The non-transitory computer readable medium of claim 28, wherein learning the class-specific sub-dictionaries and reconstruction coefficients comprises:
learning the class-specific sub-dictionaries and reconstruction coefficients under elastic-net regularization.

30. The non-transitory computer readable medium of claim 29, wherein learning the class-specific sub-dictionaries and reconstruction coefficients comprises:
iteratively optimizing an objective function by:
updating reconstruction coefficients for the local feature descriptors extracted each training image of each class with a fixed discriminative dictionary, and
updating bases in each class-specific sub-dictionary with fixed reconstruction coefficients.

31. (canceled)

32. (canceled)

33. The non-transitory computer readable medium of claim 25, wherein encoding each of the local feature descriptors into the coded local feature descriptor using the learnt discriminative dictionary comprises:
determining reconstruction coefficients to encode each of the local feature descriptors under an elastic-net regularizer using the learnt discriminative dictionary.

34. The non-transitory computer readable medium of claim 25, wherein encoding each of the local feature descriptors into the coded local feature descriptor using the learnt discriminative dictionary comprises:
determining reconstruction coefficients to encode each of the local feature descriptors by a nearest dictionary basis in the learnt discriminative dictionary.

35. The non-transitory computer readable medium of claim 25, wherein encoding each of the local feature descriptors into the coded local feature descriptor using the learnt discriminative dictionary comprises:
determining reconstruction coefficients to encode each of the local feature descriptors under a locality-constrained linear regularizer using the learnt discriminative dictionary.

36. The non-transitory computer readable medium of claim 25, wherein encoding each of the local feature descriptors into the coded local feature descriptor using the learnt discriminative dictionary comprises:
determining reconstruction coefficients to encode each of the local feature descriptors under a locality-constrained sparse regularizer using the learnt discriminative dictionary.

37. The non-transitory computer readable medium of claim 25, wherein encoding each of the local feature descriptors into the coded local feature descriptor using the learnt discriminative dictionary comprises:
determining reconstruction coefficients to encode each of the local feature descriptors under a locality-constrained elastic-net regularizer using the learnt discriminative dictionary.

38. The non-transitory computer readable medium of claim 25, wherein the operations further comprise:
repeating the steps of extracting local feature descriptors, encoding each of the local feature descriptors using the learnt discriminative dictionary, and applying the machine learning-based classifier to classify the tissue in the endomicroscopy image for each of a plurality of endomicroscopy images in an endomicroscopy video stream; and
classifying the tissue in the endomicroscopy video stream based on the classification of the tissue in each of the plurality of endomicroscopy images in an endomicroscopy video stream.

39. The non-transitory computer readable medium of claim 25, wherein the endomicroscopy image is an endomicroscopy image of a brain tumor, and applying the machine learning-based classifier to classify the tissue in the endomicroscopy image comprises:
classifying the tissue in the endomicroscopy image as glioblastoma or meningioma using the machine learning-based classifier based on the coded local feature descriptors.
